(19) United States
(12) Patent Application Publication
(10) Pub. No.: US 2024/0415751 A1
(43) Pub. Date: Dec. 19, 2024

(54) WATER REDUCED COMPACT SHAMPOO COMPOSITION

(71) Applicant: WELLA GERMANY GMBH, Darmstadt (DE)

(72) Inventors: Christian SPRINGOB, Lorsch (DE); Antonio MARTINEZ-CAMPOY, Ruesselsheim (DE); Sabine BAECKER, Ruesselsheim (DE)

(21) Appl. No.: 18/703,282

(22) PCT Filed: Oct. 21, 2022
(86) PCT No.: PCT/EP2022/079449

§ 371 Date: Apr. 19, 2024

(30) Foreign Application Priority Data
Oct. 22, 2021 (WO) PCT/EP2021/079372

(51) Int. Cl. A61K 8/34; A61K 8/46; A61Q 5/02

(52) U.S. Cl. CPC A61K 8/345; A61K 8/466; A61Q 5/02; A61K 2800/30; A61K 2800/33

(57) ABSTRACT

A shampoo composition is disclosed which comprises a combination of a surfactant component, a liquid or solid polyol and a very low concentration of water.

### FIELD OF THE INVENTION

The present invention relates to a stable, water reduced and compact shampoo composition that comprises a surfactant component comprising at least an anionic surfactant, a polyol and a very low amount of water.

### BACKGROUND OF THE INVENTION

Many consumers and stylists are requesting more sustainable products with a positive impact on carbon dioxide emission and water footprint reduction. For instance, compact/water reduced shampoo compositions can contribute to improved sustainability since conventional shampoos typically contain >75% of water and lower water levels/shampoo concentrates respectively compacted shampoos can reduce transportation weight, result in less packaging waste and an overall better carbon dioxide footprint.

Nevertheless, today's consumers do expect high performing shampoos and are typically not willing to compromise on the product performance for improved sustainability. A typical problem with water reduced, compact shampoos is the difficulty to formulate a shampoo composition that has acceptable washability/rinseability, conditioning performance, cleansing and stability, and clarity. Especially washability/rinseability speed is often a problem since water reduced, compact shampoos have a higher, pasty consistency which does need a longer, more intense and more water consuming wash-out phase. Additionally, more and more consumers prefer a shampoo composition that is free of harsh components, produces substantial lather volume and cleansing and readily dilutes and rinses with no residue.

It would therefore be of tremendous advantage to have compact, water reduced shampoo compositions delivering the same or even better washability/rinseability and product performance in comparison to conventional water-based shampoos.

### SUMMARY OF THE INVENTION

According to the present invention, these desirable attributes and properties are satisfied by an aspect of the invention comprising a compact, reduced water shampoo composition. Embodiments of the water-reduced shampoo composition according to the invention comprise at least a surfactant component, a polyol and a small amount of water. Surprisingly, it was found that a surfactant component comprising a mixture of at least two (and preferably even three) non-sulfate anionic surfactants and a non-ionic surfactant results in the formation of water-reduced shampoo composition with a high performance which is the same or even better than conventional shampoos (typically containing >75% of water), while being more sustainable and having a significantly improved ecological footprint.

The surfactant component comprises at least two non-sulfate anionic surfactants and a non-ionic surfactant. The polyol may be a per-hydric alcohol, a trihydric alcohol, a dihydric alcohol, or a combination thereof. The polyol may be liquid or solid. When the polyol is a solid it forms a dispersion or solution with a liquid polyol, a small amount of water or a combination thereof.

The embodiments of the shampoo composition allow formulation of a compact, significantly water reduced shampoo. A significantly reduced amount of water of from about one-third to one fourth of the typical amount of water of a conventional shampoo is achieved by embodiments of the water-reduced shampoo composition without the typical disadvantages of compact/water reduced shampoo relating to washability/rinseability speed, and product performance.

It was found that a water-reduced shampoo composition having a surfactant component with just one single or only two types of surfactants lack performance properties. The shampoo composition of the present disclosure comprising the combination of at least two non-sulfate anionic surfactants (and preferably even three) and a non-ionic surfactant enables excellent foaming behaviour, improved washability, and an excellent rinseability requiring less water than known shampoo compositions, while having satisfactory viscosity, and good conditioning performance. The shampoo compositions of the present disclosure have a suitable viscosity, i.e. do not have a thick and pasty consistency, and can be washed out faster and by using less water in comparison to other water-reduced shampoos.

Without being bound by theory, it is believed that the non-ionic surfactant contributes a significant part to the good foaming behaviour. Although the non-sulfate anionic surfactants also exhibit foaming properties, it has been found that the foaming properties are substantially increased by adding a non-ionic surfactant, especially when using a glucamide non-ionic surfactant. The performance can be further improved by including a third non-sulfate anionic surfactant.

The inventors of the present disclosure have further found that a high product performance can be realised—while solubilizing all components without precipitation of any component—in water-reduced shampoos by using a rather large amount of surfactant components (compared to conventional shampoos) in conjunction with a suitable solvent. The inventors have observed that a trihydric, dihydric or small polyhydric polyol (e.g. a C4 to C8 per-hydroxy alkane) in conjunction with a surfactant component concentration of at least 10 wt %, preferably at least 15 wt % or even at least 20 wt % allows for an excellent product performance and for obtaining a stable composition without causing precipitation of any component. For example, it has been observed that glycerin is particularly well suited for dissolving the rather large amount of surfactants used, while resulting in a composition which is stable over a long period of time. It was found that isethionate compounds allow to formulate stable and skin compatible compositions even at high surfactant concentrations and thus are particularly well suited as first non-sulfate anionic surfactant. The second non-sulfate anionic surfactant and the non-ionic surfactant allow for further increasing the surfactant concentration.

Furthermore, the water-reduced shampoo composition of the present disclosure are preferably essentially or completely free of a sulfate anionic surfactant. Many sulfate anionic surfactants are suspected to be environmentally harmful. Furthermore, several sulfate anionic surfactants have been reported as being irritating to the skin.

### Definitions

Unless defined otherwise, all technical and scientific terms used herein have the same meaning as commonly understood by a person of ordinary skill in the art.

As used in the specification and the appended statements, the singular forms “a,” “an” and “the” include plural referents unless the context clearly dictates otherwise.

The term “may” in the context of this application means “is permitted to” or “is able to” and is a synonym for the term “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B.

The term “about” is understood to mean ±10 percent of the recited number, numbers or range of numbers.

The term “about 0 wt %” is understood to mean that no substance, compound or material to which zero (0) refers is present, up to a negligible but detectable amount is present, assuming that the detectability can be determined on a parts per million basis.

Where features or aspects of the invention are described in terms of Markush groups, those skilled in the art will recognize that the invention is also thereby described in terms of any individual member or subgroup of members of the Markush group. For example, if X is described as selected from the group consisting of methyl, ethyl or propyl, claims for X being methyl and claims for X being ethyl and X being propyl are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “rinse-off” or “rinseability” generally includes application to the skin and/or hair followed by rinsing the product from the skin and/or hair with water within a few seconds to minutes as is typically practiced in a shower.

### BRIEF DESCRIPTION OF THE DRAWINGS

The components in the Figures are not necessarily to scale, instead emphasis being placed upon illustrating the principles of the invention. The accompanying drawings relate to embodiments of the disclosure and are described in the following:

FIG. 1 shows three images illustrating an exemplary method of applying the shampoo composition according to embodiments described herein. FIG. 1 shows ease of rinsing off the composition according to embodiments described herein.

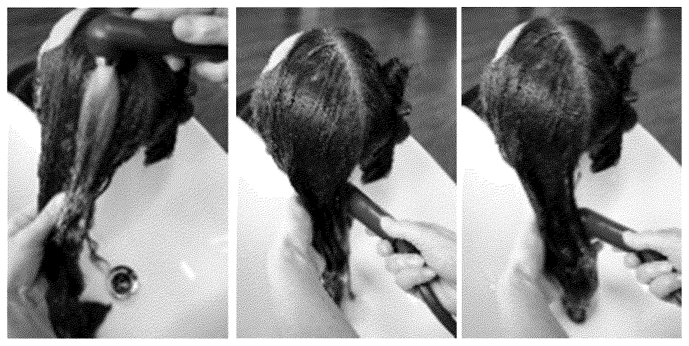

### DETAILED DESCRIPTION OF THE INVENTION

According to the invention, embodiments of the shampoo composition comprise at least (a) a surfactant component, and (b) a solid or liquid polyol. When the polyol is a solid, it may be formulated as a dispersion or solution by combination with a liquid polyol or a minor amount of water.

According to the invention, embodiments of surfactant component of the shampoo composition may comprise at least two members of the anionic surfactant class including a sulfate anionic surfactant or a non-sulfate anionic surfactant. The anionic surfactant class may also include multiple species of each member as well as a combination of these class members and any combination of multiple species of both members.

Additionally, the surfactant component further comprises a non-ionic surfactant in combination with any variation of the anionic surfactant class described above.

The surfactant component may comprise one or more sulfate anionic surfactants alone, one or more non-sulfate anionic surfactants alone, a mixture of a minor amount of one or more sulfate anionic surfactants and a major amount of one or more non-sulfate anionic surfactants, a mixture of one or more sulfate anionic surfactants and one or more non-ionic surfactants, or a mixture of one or more non-sulfate anionic surfactants and one or more non-ionic surfactants. The surfactant component may comprise one or more non-sulfate anionic surfactants alone.

Preferably, the surfactant component may comprise one or more non-sulfate anionic surfactants combined with one or more non-ionic surfactants. Especially more preferably the surfactant component may comprise a combination of two members of the surfactant class such as but not limited to a combination of one or more sulfate-free non-sulfate anionic surfactants and one or more non-ionic surfactants. Another especially preferred surfactant class may comprise any of the embodiments including a non-sulfate anionic surfactant which may be devoid of any sulfate anionic surfactant. This surfactant class may be characterized as a sulfate-free non-sulfate anionic surfactant. Without the characterization of “sulfate free”, a surfactant component comprising a non-sulfate anionic surfactant and/or a non-ionic surfactant may include but not necessarily include a minor concentration to an almost indetectable concentration of sulfate anionic surfactant which may be incidentally or purposefully present.

The sulfate anionic surfactant class of the surfactant component of the present shampoo composition may comprise a branched and/or linear C8 to C20 alkyl and/or alkenyl sulfate, a linear and/or branched C8 to C20 alkyl benzene sulfate or a linear and/or branched C8 to C20 alkyl benzyl sulfate or the corresponding ether and/or glyceryl and/or sugar alcohol derivatives obtained from ethylene epoxide/ethylene glycol, propylene epoxide/1,2-propylene glycol, glycerin and/or sugar alcohol monomers. The linear and/or branched alkyl and/or alkenyl group may be a single chain length or may be a mixture of chain lengths. The alkenyl group may have 1 or 2 sites of unsaturation. The ether and glyceryl derivatives may comprise mono, di, tri, tetra, penta monomers up to 100, preferably up to 50, more preferably up to 20 monomeric units. The sugar alcohol derivatives may comprise mono, di, tri, tetra, penta monomers up to 100, preferably up to 50, more preferably up to 20 monomeric units of a C4-C6 sugar alcohol including but not limited to examples such as erythritol, xylitol, mannitol, sorbitol, isomalt and maltitol. The counterion of the sulfate anionic surfactant may be an alkali metal, ammonia and/or a primary, secondary or tertiary C1-C6 alkyl amine or aminoalcohol such as but not limited to ammonium cation, sodium, potassium, trimethyl amine, triethyl amine, ethanolamine, triethanolamine, diethanolamine.

The sulfate anionic surfactant class may be represented by the chemical formulas: ROSO₃M, RC₆HOSO₃M, RC₆H₄CH₂OSO₃M, RO(C₂H₄O)ₓOSO₃M, RO(C₂H₄O)ₓC₆H₄OSO₃M, RO(C₂H₄O)ₓC₆H₄CH₂OSO₃M, RO(CH₂CH (Me)O)ₓOSO₃M, RO(CH₂CH (Me)O)ₓC₆H₄OSO₃M, RO(CH₂CH(Me)O)ₓC₆H₄CH₂OSO₃M, RO(CH₂CHOHCH₂O)ₓOSO₃M, RO(CH₂CHOHCH₂O)ₓC₆H₄OSO₃M, RO(CH₂CHOHCH₂O)ₓC₆H₄CH₂OSO₃M, RO(sugar alcohol)ₓOSO₃M, RCO₂(C₂H₄O)ₓOSO₃M, RCO₂ (C₂H₄O)ₓC₆H₄OSO₃M, RCO₂ (C₂H₄O)ₓC₆H₄CH₂OSO₃M, RCO₂ (CH₂CH (Me)O)ₓOSO₃M, RCO₂ (CH₂CH (Me)O)ₓC₆H₄OSO₃M, RCO₂ (CH₂CH (Me)O)ₓC₆H₄CH₂OSO₃M, RCO₂ (CH₂CHOHCH₂O)ₓOSO₃M, RCO₂ (CH₂CHOHCH₂O)ₓC₆H₄OSO₃M, RCO₂ (CH₂CHOHCH₂O)ₓC₆H₄CH₂OSO₃M, RCO₂ (sugar alcohol)ₓOSO₃M. The R group of these formulas may be a linear or branched C8 to C20 alkyl and/or alkenyl group derived from a fatty alcohol and may be a single chain length or may be a mixture of chain lengths. The RCO₂ group is a fatty acid derivative corresponding to the fatty alcohol derivative RO—. The designator x of the ether/polyether/polyglyceryl/polysugar alcohol unit may be an integer of from 1 to 100, preferably up to 50, more preferably up to 20. Me is methyl and M is a counterion as described above. Preferred members of this class include the linear and/or branched sulfate anionic surfactants of the formulas ROSO₃M, RC₆HOSO₃M, RO(C₂H₄O)ₓOSO₃M and/or RO(CH₂CHOHCH₂O)ₓOSO₃M. The preferred alkyl/alkenyl group of these preferred members may be a C8 to C20 linear or branched alkyl group or a C14 to C20 linear or branched alkenyl group or mixtures of alkyl and/or alkenyl lengths or mixtures of alkyl and alkenyl groups. More preferably, the alkyl group may be a C10 to C18 linear or branched single length alkyl or mixture of lengths and the alkenyl group may be a C14 to C20 linear single length alkenyl group and/or a mixture thereof. Additionally included are mixtures of alkyl and alkenyl groups. Especially more preferably the alkyl and alkenyl group may be capryl, undecyl, lauryl, myristyl, palmityl, cetyl, stearyl oleyl, myristolyl, palmitolyl, sapienyl or mixtures thereof such as cocoyl which is a mixture of alkyl groups derived from coconut oil and includes caprylic, capric, lauric, myristic, palmitic and oleic groups.

Exemplary sulfate anionic surfactants comprise one or more of sodium lauryl sulfate, sodium laureth sulfate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric mono glyceride sodium sulfate, sodium lauryl sulfate, sodium laureth sulfate, potassium lauryl sulfate, potassium laureth sulfate, ammonium cocoyl sulfate, sodium cocoyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfate, sodium dodecyl benzene sulfate.

The non-sulfate anionic surfactant class—i.e. the first, second, and optional third or even an optional fourth sulfate-free anionic surfactant—of the surfactant component of the present shampoo composition may be selected from the group of acyl isethionates/methyl isethionates, acyl glycinates, acyl taurates, acyl amino acids, acyl sarcosinates, sulfosuccinates and sulfonates, wherein the acyl groups of all these surfactant classes comprise from 6 to 30 carbon atoms. The non-sulfate anionic surfactant may also comprise more than one of these surfactants and may comprise mixtures thereof. The non-sulfate anionic surfactant segment may also comprise the alkaline and alkaline earth salts of one or more or mixtures of individual compounds of any of these surfactant classes and mixtures thereof.

One member of the non-sulfate anionic surfactant class of the surfactant component of the present shampoo composition according to the invention may comprise acyl isethionates/methyl isethionates. This class comprises fatty acid esters of 2-hydroxyethyl sulfonate and 2-hydroxy-2-methylethyl sulfonate. These acyl isethionates/methyl isethionates have the respective formulas RCO₂CH₂CH₂SO₃X and RCO₂CH(CH₃)CH₂SO₃X. The substituent X may be hydrogen or an alkaline or alkaline earth metal. The acyl group may be a saturated or unsaturated group RCO— of 6 to 30 carbons in which R is a linear or branched, preferably linear saturated alkyl group of 5 to 29 carbons and/or is a linear unsaturated alkyl group of 5 to 29 carbons and 1 to 3 unsaturation sites. In other words, the acyl group, RCO—, corresponds to the acyl derivative of a C6-C30 fatty acid, including but not limited to a C6-C30 fatty linear or branched, preferably linear alkyl carboxylic acid and/or a C6-C30 alkenyl carboxylic acid with 1, 2 or 3 unsaturation sites in which the carboxylic acid group of the alkyl carboxylic acid and the alkenyl carboxylic acid is positioned at a terminus.

Exemplary acyl isethionates and methyl isethionates are esters formed with isethionic acid and/or methyl isethionic acid and one or more C6-C30 fatty acids. Particular embodiments of the acyl group, RCO—, derived from a C6-C30 fatty acid may be selected from saturated acyl groups including but not limited to hexoyl, heptoyl, capryloyl, caproyl, lauroyl, myristoyl, palmitoyl (cetoyl), stearoyl, arachidoyl, behenoyl, lignoceroyl, cerotoyl, nonoyl, undecoyl, tridecoyl, pentadecoyl, heptadecoyl, nonadecoyl; and from unsaturated acyl groups including but not limited to myristoyl, palmitolyl, sapienoyl, oleyl, elaidoyl, vaccenoyl, linolenoyl, linoelaidoyl, arachidonoyl, eicosapentaenoyl, erucoyl, docosahexaenoyl group and any combination of saturated and/or unstaturated groups thereof. While the trivial name acyl groups are derived from natural fatty acids, the systemically nomenclature acyl groups such as nonoyl, undecoyl, tridecoyl, pentadecoly, heptadecoyl, nonadecoyl are semi-synthetic fatty acids which may be derived from petroleum sources.

Fatty acid mixtures derived from vegetable oils may also be used to form this class of isethionate and methyl isethionate esters. The fatty acid mixtures may include mixtures of one or more of the single C6-C30 fatty acids, selected embodiments of which correspond to the above named acyl groups. Exemplary vegetable fatty acid mixtures include those derived from coconut oil, palm oil, olive oil, sunflower oil, peanut oil, cottonseed oil, corn oil, safflower oil, flax oil, and similar vegetable oils. The resulting exemplary fatty acid mixtures include but are not limited to coconut fatty acids, palm fatty acids, olive fatty acids, corn oil fatty acids, safflower fatty acids, sunflower fatty acids, flax fatty acids and similar vegetable fatty acids. These fatty acid mixtures esterify the isethionic acid and/or methyl isethionic acid to form the fatty acyl isethionate and/or acyl methyl isethionate ester mixtures.

The preferable isethionate and/or methyl isethionate members of this class of the surfactant component according to the invention comprises an isethionate/methyl isethionate selected from the group of sodium cocoyl isethionate, sodium cocoyl methyl isethionate, sodium lauroyl isethionate, sodium lauroyl methyl isethionate and sodium oleoyl methyl isethionate. Of these preferred isethionate/methyl isethionates, sodium cocoyl isethionate and sodium cocoyl methyl isethionate are especially preferred.

Another member of the non-sulfate anionic surfactant class of the surfactant component of the present shampoo composition may comprise N-acyl glycinates and N-acyl amino acids and their salts. This member includes C6-C30 fatty acid amides of the amino acid, glycine, as well as the C6-C30 fatty acid amides of other natural and synthetic amino acids, and the alkaline and alkaline earth metal salts thereof. The N-acyl glycinates have the formula RCO—NHCH₂CO₂X in which X is hydrogen or an alkaline or alkaline earth metal. The N-acyl amino acids have the formula RCO—NH(CH₂)ₙCHR′CO₂X in which R′ is any of the other natural and well-known synthetic amino acid substituents, n is zero or an integer of 1 to 4, and X is hydrogen or an alkaline or alkaline earth metal. Particular embodiments of the N-acyl amino acids provide R′ as the α-substituent of a natural amino acid such as alanine, valine, methionine, phenyl alanine, threonine, serine, asparagine, tyrosine, lysine. Preferred as the amino acid portion of the N-acyl amino acid are alanine, phenyl alanine, threonine, serine and glutamine. In addition, for synthetic amino acids, R′ may be hydrogen as well as the α-substituent of a natural amino acid and n is an integer of 1 to 4. Preferable synthetic amino acids include those in which R′ is hydrogen and n is 1 or 2.

The acyl group, RCO—, of N-acyl glycinate and N-acyl amino acids of the foregoing formulas may comprise the fatty acyl group RCO— as described above for the acyl isethionate/methyl isethionate class. The acyl group may be a single acyl group or it may comprise a mixture of acyl groups as described above for the acyl isethionate/methyl isethionate class. The mixture may be derived from vegetable fatty acids as described above for the isethionate/methyl isethionate class.

Another member of the non-sulfate anionic surfactant class of the surfactant component of the present shampoo composition may comprise the class of acyl taurates and acyl sarcosinates. This class includes N-fatty acyl taurate salts or N-fatty acyl sarcosinate salts, the N-alkyl derivatives thereof or any combination thereof. The taurate and sarcosinate surfactants have the following formulas wherein RCO— represents the fatty acyl substituent, R′ is hydrogen or alkyl and M represents an alkaline or alkaline earth metal, preferably including sodium and potassium, especially sodium.

RCO—NR′CH₂CH₂SO₃M N-fatty acyl sarcosinate salt

RCO—NR′CH₂CO₂M N-fatty acyl taurate salt

The N-fatty acyl group for the taurate salt may be any longer chain saturated or unsaturated fatty group. Preferably, the fatty acyl group for the taurate salt is an unsaturated fatty acyl group of 6 to 30 carbons, preferably 12 to 22 carbons, more preferably 14 to 20 carbons, especially more preferably 14 to 18 carbons including myristoyl, palmitolyl, sapienoyl, oleyl, elaidoyl and vaccinoyl acyl groups, most preferably oleyl and palmitoyl acyl groups, especially most preferably the oleyl group. The N-alkyl group is a C1 to C6 linear or branched alkyl group, preferably methyl, ethyl, propyl or isopropyl and more preferably methyl.

The N-fatty acyl group for the sarcosinate salt may be any shorter chain saturated fatty group. Preferably the fatty acyl group for the sarconinate salt is a saturated fatty acyl group of 6 to 30 carbons, especially preferably 6 to 14 carbons including caproloyl, enanthoyl, capryloyl, pelargonoyl, caproyl, undecyloyl, lauroyl, tridecyloyl and myristoyl groups, more preferably 8 to 12 carbons, and most preferably the lauroyl C12 acyl group. The N-alkyl group is the same as that for the taurate salt except that R′ as hydrogen is excluded. R′ for the sarconsinate salt is preferably methyl.

Another member of the non-sulfate anionic surfactant class of the surfactant component of the present shampoo composition may comprise the class of sulfosuccinates and sulfosulfonates. The which encompass alkyl sulfosuccinates, polyalkoxylated sulfosuccinates, and alkylamide sulfosuccinates and the sulfosulfonates thereof wherein the alkyl group may be a C6-C30 alkyl group and the polyalkoxylate group may be formed of from 2 to 30 units of an ethylene or propylene glycol monomer. Exemplary alkyl, polyalkoxylated and alkylamide sulfosuccinates and sulfosulfonates include disodium laureth sulfosucciniate and sulfosulfonate, disodium lauryl sulfosuccinate and sulfosulfonate, disodium oleth-3 sulfosuccinate and sulfosulfonate, disodium deceth-6 sulfosuccinate and sulfosulfonate, disodium polyethylene glycol (10) sulfosuccinate and sulfosulfonate, disodium polypropylene glycol (10) sulfosuccinate and sulfosulfonate, disodium cetylamide sulfosuccinate and sulfosulfonate, disodium stearylamide sulfosuccinate and sulfosulfonate, disodium palmylamide sulfonsuccinate and sulfosulfonate and disodium dodecylamide sulfosuccinate and sulfosulfonate.

When a single member of the class of non-sulfate anionic surfactant is to be incorporated as the corresponding surfactant component, the preferred non-sulfate anionic surfactant member comprises acyl isethionates/methyl isethionates. With this preferred arrangement, the other members of the non-sulfate anionic surfactant class may be added to the acyl isethionates/methyl isethionates to provide a mixture of members of non-sulfate anionic surfactant class. It is preferred that the majority member of this mixture of members be the acyl isethionates/methyl isethionates. More preferably, the acyl isethionate/methyl isethionate relative to the other non-sulfate anionic surfactant members may be in a wt % ratio of from about 19:3 to about 17:6, preferably about 19:3.5 to about 17:6, more preferably about 18.5:3.5 to about 17.4:5.8.

The non-ionic surfactant class of the surfactant component of the shampoo composition according to the invention comprises the classes of acyl-amino deoxy sugars, acyl-amino sugar alcohols and alkyl glycosides. The acyl amino deoxy sugars and acyl amino sugar alcohols alcohols have the formula R¹—CO—NX—R² wherein R¹—CO is a linear or branched C6-C30 fatty acyl group with R¹ being a linear or branched C5-C29 alkyl group. R² is hydrogen or C1-C6 alkyl. The moiety —NX is amino deoxy sugar or amino sugar alcohol. The amino deoxy sugar group has the open form formula

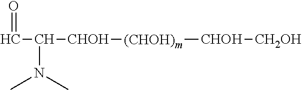

with m as zero or 1. The amino sugar alcohol group has the formula ═N—CH₂—CHOH—CHOH—(CHOH)ₘ—CHOH—CH₂OH with m being zero or 1. Examples of amino sugars and amino sugar alcohols include the —NH₂ derivatives of deoxy pentose, deoxy hexose, di-deoxyhexose and the corresponding alcohols. The corresponding deoxy sugar core examples include but are not limited to deoxyglucose, deoxymannose, deoxyidose, deoxytalose, deoxygalactose, deoxyribose deoxyxylose, doxyarabinose, deoxylyxose; and sugar alcohol core examples include but are not limited to xylitol, ribitol, mannitol, sorbitol, galactitol, fucitol, iditol, inositol, volemitol, isomalt, maltitol, lactitol. Preferable amino deoxy sugars and amino sugar alcohols include glucosamine, amino deoxy mannose, amino deoxy galactose, aminosorbitol, aminomannitol and amino deoxyribose. More preferable is glucosamine. The preferable acyl-amino deoxysugars include lauroyl/myristyl methyl glucamide, capryloyl/caproyl methyl glucamide, capryloyl/caproyl glucamide.

The alkyl glycoside class of non-ionic surfactant serving as a component of the shampoo composition according to the invention comprises C8-C30 linear and branched alkyl glycosides. The glycoside cores of this class includes but is not limited to sugar moieties such as glucosides, fructosides, glucuronides, mannosides, galactosides, ribosides as well as polyglycosides such as polyglucoside, polyfructoside, polymannoside, polygalactoside and polyglucuronic acid. The alkyl group is derived from a C8-C30 fatty alcohol and forms a glycosidic bond with the anomeric carbon (the carbonyl carbon) of the sugar through formation of a ketal group from the carbonyl group. Exemplary glycosides include but are not limited to lauryl glucoside, decyl glucoside, coco glucoside, octyl glucoside, capryl glucoside, caprylyl/capryl glucoside, decyl polyglucoside, coco polyglucoside, lauryl polyglucoside and mixtures thereof.

The polyol component of the present shampoo composition may be any one or more embodiments of a polyhydric, trihydric and/or dihydric alcohol (i.e., a per-hydroxy alkane, a triol or a diol) or a dimer, trimer tetramer, pentamer thereof formed by condensing multiple hydroxy groups to form ether groups such as is formed by condensing two ethylene glycols to form diethylene glycol. The polyol may be liquid or solid. When the polyol is a solid at ambient conditions, it may be diluted with a minor amount of water or a liquid polyol to produce a flowable polyol mixture or solution. A polyol may alternatively be pasty at ambient conditions and may be combined with a liquid polyol to provide a flowable mixture or solution. The polyhydric alcohol may be a C4-C8 alkane with each carbon substituted by a hydroxyl group or a dimer or trimer thereof. The trihydric alcohol may be a C3 to C8 alkane substituted by three hydroxyl groups or a dimer or trimer thereof. The dihydric alcohol may be a C2 to C8 alkane substituted by two hydroxyl groups or a dimer or trimer thereof. Preferably, the trihydric and dihydric alcohols in pure form and without dilution with water display liquid and/or flowable properties at ambient conditions. Exemplary polyhydric alcohols include sugar alcohols such as but not limited to erythritol, xylitol and sorbitol. Exemplary triols may be selected from propylene triol (glycerin), 1,2,3-butylene triol, 1,2,5-pentylene triol and/or 1,3,6-hexylene triol and any combination thereof. Exemplary diols may be selected from ethylene glycol, 1,2-propylene diol, 1,3 propane diol, 1,2- and 1,3-butane diol, 1,2 pentane diol, diethylene glycol, dipropylene glycol and any combination thereof. Preferred polyols include sorbitol, glycerin (propyl triol), 1,2,3-butanetriol, ethylene glycol and diethylene glycol and any combination thereof. A more preferred polyol is sorbitol, glycerin, 1,2 propylene diol, 1,3 propylene diol and any combination thereof.

Embodiments of the shampoo composition according to the invention incorporate weight percentage ranges relative to the total weight of the composition of the surfactant component, the polyol and water to provide the desirable properties of the shampoo composition. The weight percentage of the surfactant component relative to the total weight of the shampoo composition may be in a range of from about 5 wt % to about 45 wt %, preferably from about 10 wt % to about 40 wt % and the surfactant component may comprise at least one anionic surfactant. When the surfactant component comprises one or more sulfate anionic surfactants alone, the surfactant component may have a concentration in the range of from about 5 wt % to about 35 wt %, preferably about 10 wt % to about 35 wt %, more preferably about 15 wt % to about 35 wt % relative to the total weight of the shampoo composition. When the surfactant component comprises one or more non-sulfate anionic surfactants alone, the surfactant component may have a concentration in the range of from about 10 wt % to about 45 wt %, preferably about 15 wt % to about 35 wt % relative to the total weight of the composition. When the surfactant component comprises a mixture of one or more sulfate anionic surfactants and one or more non-sulfate anionic surfactant, the sulfate anionic surfactant comprises no more than 10 wt %, preferably no more than about 5 wt %, more preferably no more than about 2 wt % of the total weight of the surfactant component. When the surfactant component comprises a combination of the anionic surfactant and the non-ionic surfactant wherein the anionic surfactant comprises one or both of the sulfate anionic surfactant and the non-sulfate anionic surfactant, the weight ratio of anionic surfactant to non-ionic surfactant may be in a range of about 5:1 to about 15:1, preferably about 6:1 to about 12:1, more preferably about 7:1 to about 12:1, especially more preferably about 8:1 to about 12:1. When the surfactant component comprises a combination of anionic surfactant and non-ionic surfactant, the weight percentage of the surfactant component may be in a range of about 5 wt % to about 45 wt %, preferably about 10 wt % to about 40 wt % and the weight percentage ratio of anionic surfactant to non-ionic surfactant providing the concentration of non-ionic surfactant relative to the weight percentage ranges given above for the sulfate anionic surfactant and the non-sulfate anionic surfactant. For the combination of non-sulfate anionic surfactant and non-ionic surfactant as the surfactant component, the concentration of the surfactant component may range from 10 wt % to about 40 wt %, preferably from about 15 wt % to about 35 wt %, more preferably about 20 wt % to about 30 wt %, especially more preferably about 20 wt % to about 26 wt %.

The weight ratio of a polyol or mixture thereof to water is in a weight percentage range of about 3:1 to 16:1, preferably 3:1 to 10:1, most preferably 3:1 to 5:1. The polyol of the polyol to water ratio may be any of the polyols described above, preferably the polyol for this ratio range is a trihydric alcohol, more preferably glycerol. The weight percentage of polyol relative to the weight of the composition ranges from about 45 wt % to about 80 wt %, preferably about 50 wt % to about 75 wt %, more preferably about 55 wt % to about 70 wt %, especially more preferably about 55 wt % to about 65 wt %. The weight percentage of water relative to the weight of the composition ranges from about 5 wt % to about 25 wt %, preferably about 10 wt % to about 20 wt %, more preferably about 13 wt % to about 17 wt %, most preferably 14 wt % to about 17 wt %.

The compositional embodiments can but not necessarily include optional components such as conditioning agents, antidandruff agents and direct hair dyes. Selection of the optional components is made based on their compatibility with the surfactant component and the polyol.

One optional component of the shampoo composition comprises a conditioning agent. Examples of the conditioning agent include cationic surfactants such as quaternary ammonium organic compounds as well as corresponding tertiary amine compounds, amidoammonium compounds as well as corresponding tertiary amine compounds, mono, di and polysaccharide with quaternary ammonium or tertiary amine groups, or any mixture thereof. Non-limiting classes of these cationic surfactants include mono or di fatty alkyl or alkenyl quaternary ammonium and tertiary amine compounds, mono or di polyol quaternary ammonium and tertiary amine compounds, mono or di fatty alkyl or alkenyl benzyl quaternary ammonium and tertiary amine compounds, fatty alkyl or alkenyl aryl, polyol quaternary ammonium and tertiary amine compounds, polyol mono, di or tri saccharide alkyl quaternary ammonium and tertiary amine compounds, tertiary or quaternized aminoalkyl fatty alkyl or alkenyl ester or amide compounds, phenoxyalkyl fatty alkyl or alkenyl quaternary ammonium and tertiary amine compounds, hydrolyzed starch with terminally quaternized ammonium and tertiary amine compounds, multipolyol cellulose with terminal quaternized ammonium and tertiary amine compounds, sucrose, lactose mono and disaccharides, arabic, ghatti, guaicum, guar, karaya, locust bean and xanthan gums derivatized with terminal quaternized ammonium and tertiary amine compounds, and similar hydrophobic tail with cationic head organic compounds. The classes of cationic surfactants with tertiary amine groups may be protonated by the media or by organic acid to form cationic groups.

Examples of these cationic surfactants may be selected from and/or include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, benzalkonium chloride, benzodidecinium bromide, cetalkonium chloride, dicedyldimenthlammonium chloride, dimethyldioctadecylamminum chloride, dioleoyl-3-trimethyl ammonium propane, lauryl methyl gluceth-10 hydroxylpropyl dimonium chloride, N-oleyl-1,3-propane diamine, stearalkonium chloride and mixtures thereof.

The conditioning agent comprising a cationic surfactant may be optionally included in the shampoo composition of the invention within a weight percentage range of from about 0.05 wt % to about 10 wt %, preferably from about 0.1 wt % to about 5 wt % relative to the total weight of the shampoo composition.

Additional embodiments of the shampoo composition include another optional component which is an anti-dandruff agent. Examples of the anti-dandruff agent include ketoconazole, climbazole, zinc pyrithione selenium disulfide, coal tar or sulfur. The optional anti-dandruff agent may be included in the shampoo composition of the invention within a weight percentage range of from about 0.1 wt % to about 5 wt %, preferably about 0.1 wt % to about 2 wt % relative to the total weight of the shampoo composition.

Another optional component for inclusion in embodiments of the conditioner composition includes direct dyes to provide coloring benefits. The technology of the present invention is suitable for nonionic direct dyes, especially for nonionic nitrobenzene derivatives and anionic direct dyes.

Examples of nitrobenzene derivatives include but are not limited to, for example, 4-hydroxypropylamino-3-nitrophenol, 1-amino-5 chloro-4-[(2,3-dihydroxypropyl)amino]-2-nitrobenzene (HC Red No. 10), and 5-chloro-1,4-[di-(2,3-dihydroxypropyl)amino]-2-nitrobenZene (HC Red No. 11) or 4-hydroxypropylamino-3-nitrophenol. Nonionic nitro dyes useful herein include, for example, 1,4-bis-[(2-hydroxyethyl)amino]-2-nitrobenzene, 1-(2-hydroxyethyl)amino-2-nitro-4-[di-(2-hydroxyethyl)amino]benzene (HC Blue No. 2), 1-amino-3-methyl-4-[(2-hydroxyethyl)amino]-6-nitrobenzene (HC Violet No. 1), 2-amino-4,6-dinitrophenol, 1,4-diamino-2-nitrobenzene (C176070), 4-amino-2-nitrodiphenylamine (HC Red No. 1), 1-amino-4-[di-(2-hydroxyethyl)amino]-2-nitrobenzenehydrochloride (HC Red No. 13), 1-amino-5-chloro-4-[(2-hydroxyethyl)amino]-2-nitrobenzene, 4-[(2-hydroxyethyl)amino]-3-nitrophenol, 4-[(2-nitrophenyl)amino]phenol (HC Orange No. 1), 1-[(2-amino ethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-2-[(2-hydroxyethyl)amino]-5 nitrobenzene (HC Yellow No. 5), 1-(2-hydroxyethoxy)-2-[(2 hydroxyethyl)amino]-5-nitrobenZene (HC Yellow No. 4), 1-[(2-hydroxyethyl)amino]-2-nitrobenZene (HC Yellow No. 2), 2-[(2-hydroxyethyl)amino]-1-methoxy-5-nitrobenzene as well as similar variations and derivatives thereof as known in the dye literature.

Other nonionic direct dyes useful herein include, for example, 1,4-di-[(2,3-dihydroxypropyl)amino]-9,10-anthraquinone, 1,4-di-[(2-hydroxyethyl)amino]-9,10-anthraquinone (C161545, Disperse Blue 23), 1-amino-4-hydroxy-9,10-anthraquinone (C160710, Disperse Red 15), 1-hydroxy-4-[(4-methyl-2-sulfophenyl)amino]-9,10-anthraquinone, 7-beta-D-glucopyranosyl-9,10-dihydro-1-methyl-9,10-dioxo-3,5,6,8-tetrahydroxy-2-anthracenecarboxylic acid (C175470, Natural Red 4), 2-((4-(di-(2-hydroxyethyl)amino)phenyl)amino)-5-((2-hydroxyethyl)amino)-2,5-cyclohexadien-1,4-dione (HC Green No. 1), 2-hydroxy-1,4-naphthoquinone (C175480, Natural Orange No. 6), 1,2-dihydro-2-(1,3-dihydro-3-oxo-2H-indol-2-ylidene)-3H-indol-3-one (C173000), 1-[di-(2-hydroxyethyl)amino]-4-[(4-nitrophenyl) azo]benzene, (Disperse Black No. 9), 4-[(4-aminophenyl) azo]-1 [di-(2-hydroxyethyl)amino]-3-methylbenzene (HC Yellow No. 7), 2), tri-(4-amino-3-methylphenyl) carbenium chloride (C142520; Basic Violet No. 2), 1-[(4-aminophenyl) azo]-7 (trimethylammonio)-2-naphthol chloride (C112250; Basic Brown No. 16), 3-[(4-amino-2,5-dimethoxyphenyl) azo]-N,N,N-trimethylbenzolaminium chloride (C11 12605, Basic Orange No. 69), 1-[(4-amino-2-nitrophenyl) azo]-7-(trimethylammonio)-2-naphthol chloride (Basic Brown No. 17), and 1-methyl-4-((methylphenyl-hydrazono)methyl)pyridinium methyl sulfate (Basic Yellow No. 87).

Other cationic direct dyes useful herein include, for example, Benzenamine, 4-[(2,6-Dichlorophenyl) (4-amino-3,5-Dimethyl-2,5-Cyclohexadien-1-ylidene)Methyl]-2,6 Dimethyl-, Phosphate) (HC Blue No. 15), 1-(2-morpholiniumpropylamino)-4-hydroxy-9,10-anthraquinone methylsulfate, and 1-[(3-(dimethylpropylaminium)propyl)amino]4-(methylamino)-9,10-anthraquinone chloride.

Anionic direct dyes useful herein include, for example, disodium bis [4-(N-ethyl-N-3-sulfonatophenylmethyl)aminophenyl]phenylmethylium (INCI name: Acid Blue 9), Benzenesulfonic acid, 2-[(9,10-Dihydro-4-hydroxy9,10-Dioxo-1-anthracenyl)amino]-5-methyl-, monosodiumsalt (INCI name: Ext. Violet 2), p-((2-Hydroxy-1-naphthyl)azo)benzenesulfonic acid sodium salt (INCI name: Orange 4), 2,2′-(1,4-Anthraquinonylenediimino)bis(5-methylbenzenesulfonic acid) disodium salt (INCI name: ACID GREEN 25), Acides 2-(2-quinoleyl) 1,3-indanedione mono, di, trisulfoniques, sodium salt (INCI name: Yellow 10), 5-amino-4-hydroxy-3-0-(phenylazo)-2,7-naphthalenesulfonic acid, disodium salt (INCI name: Acid Red 33).

According to another aspect, the present invention relates to a method of shampooing and conditioning hair through use of compositional embodiments of the invention. Embodiments of the method involve application of the shampoo composition to hair followed with or simultaneous with dilution with water.

Aspects and embodiments of the invention may be defined by the following clauses.
Clause 1. A water-reduced shampoo composition comprising:
i) a liquid polyol at a weight percentage concentration of from about 40 wt % to about 80 wt %, preferably from about 45 wt % to about 80 wt %;
ii) a surfactant component at a weight percentage concentration of from about 5 wt % to about 45 wt %, preferably from about 10 wt % to about 40 wt %, more preferably from about 15 wt % to about 35 wt %; and the surfactant composition comprises at least one anionic surfactant;
iii) water at a weight percentage concentration of from about 5 wt % to about 25 wt %; wherein all weight percentages are relative to the total weight of the composition.
Clause 2. A water-reduced shampoo composition according to clause 1 wherein the surfactant component further comprises at least one or more non-ionic surfactants.
Clause 3. A water-reduced shampoo composition according to clause 1 or 2 wherein the surfactant component has a concentration of from about 5 wt % to about 35 wt %, preferably about 10 wt % to about 35 wt %, more preferably about 15 wt % to about 35 wt % relative to the total weight of the shampoo composition and comprises at least one or more sulfate anionic surfactants.
Clause 4. A water-reduced shampoo composition according to clause 1 or 2 wherein the surfactant component is essentially or completely free of a sulfate anionic surfactant, has a concentration of from about 10 wt % to about 40 wt %, preferably about 15 wt % to about 35 wt % relative to the total weight of the shampoo composition and comprises at least one or more sulfate-free anionic surfactants.
Clause 5. A water-reduced shampoo composition according to clause 1, 2 or 3 wherein the surfactant component comprises at least a mixture of a sulfate anionic surfactant and a sulfate-free anionic surfactant; and preferably the sulfate anionic surfactant comprises no more than 10 wt %, preferably no more than 5 wt %, more preferably no more than 2 wt % of the total wt % of the mixture of the sulfate anionic surfactant and the sulfate-free anionic surfactant.
Clause 6. The water-reduced shampoo composition of any of the preceding clauses, wherein the surfactant component comprises at least an anionic surfactant and a non-ionic surfactant and the weight percentage ratio of anionic surfactant to non-ionic surfactant is about 5:1 to about 15:1, preferably about 8:1 to about 12:1.
Clause 7. The water-reduced shampoo composition of any of the preceding clause wherein the anionic surfactant comprises one or more of sodium lauryl sulfate, sodium laureth sulfate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric mono glyceride sodium sulfate, sodium lauryl sulfate, sodium laureth sulfate, potassium lauryl sulfate, potassium laureth sulfate, ammonium cocoyl sulfate, sodium cocoyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfate, sodium dodecyl benzene sulfate; preferably the anionic surfactant comprises one or more of sodium cocoyl isethionate, sodium cocoyl methyl isethionate, sodium lauroyl isethionate, sodium lauroyl methyl isethionate, sodium lauroyl sarcosinate, sodium methyl oleoyl taurate, and any combination thereof; and more preferably the anionic surfactant comprises an isethionate compound at a weight percentage range of from about 5 wt % to about 25 wt % relative to the total weight of the composition.
Clause 8. The water-reduced shampoo composition of any of the preceding clause, wherein the non-ionic surfactant comprises one or more of lauroyl/myristyl methyl glucamide, capryloyl/caproyl methyl glucamide, capryloyl/caproyl glucamide, lauryl glucoside, decyl glucoside, coco glucoside, and any combination thereof.
Clause 9. The water-reduced shampoo composition of any of the preceding clause, wherein the composition is substantially free of amphoteric surfactants.
Clause 10. The water-reduced shampoo composition of any of the preceding clauses wherein the polyol comprises at least a polyhydric, trihydric or dihydric alcohol, a dimer or trimer thereof, or any combination thereof; preferably the polyhydric alcohol is a C4 to C8 per-hydroxy alkane, the trihydric alcohol is a trihydroxyl C3-C8 alkane. a dimer or trimer thereof and the dihydric alcohol is a dihydroxyl C2-C8 alkane, a dimer or trimer thereof; and any combination of the perhydroxy alkane, trihydric alcohol and dihydric alcohol; more preferably the polyol is erythritol, xylitol, sorbitol, propylene triol, 1,2,3-butylene triol, 1,2,5-pentylene triol, 1,3,6-hexylene triol, ethylene glycol, 1,2-propylene diol, 1,3 propane diol, 1,2-butane diol, 1,3-butane diol, 1,2 pentane diol, diethylene glycol, dipropylene glycol and any combination thereof; especially more preferably the polyol comprises sorbitol, glycerin (propyl triol), 1,2,3-butanetriol, ethylene glycol, diethylene glycol, 1,2 propylene diol, 1,3 propylene diol and any combination thereof; most preferably the polyol comprises sorbitol, glycerin, 1,2-propanediol, 1,3 propanediol and any combination thereof; and especially most preferably the polyol is glycerin.
Clause 11. The water-reduced shampoo composition of any of the preceding clauses, wherein the composition further comprises a conditioning agent; preferably the concentration of the conditioning agent is in a range of from about 0.05 wt % to about 10 wt %, more preferably from about 0.1 wt % to about 5 wt % relative to the total weight of the shampoo composition; and especially more preferably the conditioning agent comprises at least a quaternary ammonium organic compound.
Clause 12. The water-reduced shampoo composition of any of the preceding clauses, wherein the composition further comprises an anti-dandruff agent selected from the group consisting of climbazole, ketoconazole, zinc pyrithione, selenium disulfide and mixtures thereof.
Clause 13. The water-reduced shampoo composition of clause 12, wherein the concentration of the anti-dandruff agent is in a range of about 0.1 wt % to about 5 wt %, preferably 0.1 wt % to about 2 wt % relative to the total weight of the shampoo composition.
Clause 14. The water-reduced shampoo composition of any of the preceding clauses, wherein the composition further comprises a direct dye selected from the group consisting of coal tar or sulfur dyes, azo dyes, anthraquinone dyes, cationic dyes, anionic dyes and mixtures thereof.
Clause 15. Use of the water-reduced shampoo composition of any of the preceding clauses for reducing transportation weight and/or reducing packaging waste.
Clause 16. A water-reduced shampoo composition comprising:
i) a liquid polyol at a weight percentage concentration of from about 40 wt % to about 80 wt %, wherein the polyol comprises at least a polyhydric, trihydric or dihydric alcohol, a dimer or trimer thereof, or any combination thereof; the polyhydric alcohol being a C4 to C8 per-hydroxy alkane; the dihydric alcohol being a dihydroxyl C2-C8 alkane;
ii) a surfactant component at a weight percentage concentration of from about 10 wt % to about 40 wt %
wherein the surfactant component further comprises at least one or more non-ionic surfactants;
wherein the surfactant component further comprises at least a first sulfate-free anionic surfactant, and a second sulfate-free anionic surfactant; and wherein the first sulfate-free anionic surfactant and the second sulfate-free anionic surfactant are different components;
iii) water at a weight percentage concentration of from about 5 wt % to about 25 wt %; wherein all weight percentages are relative to the total weight of the composition.
Clause 17. The water-reduced shampoo composition of clause 16 wherein the polyol comprises a trihydric alcohol, and wherein the trihydric alcohol is a trihydroxyl C3-C8 alkane.
Clause 18. The water-reduced shampoo composition of clause 17, wherein the trihydric alcohol is glycerin.
Clause 19. The water-reduced shampoo composition of any one of clauses 16-18 wherein the first sulfate-free anionic surfactant is an isethionate compound at a weight percentage range of from about 5 wt % to about 25 wt % relative to the total weight of the composition.
Clause 20. The water-reduced shampoo composition of clause 19 wherein the first sulfate-free anionic surfactant is sodium cocoyl isethionate.
Clause 21. A water-reduced shampoo composition of any of any one of clauses 16-20 wherein the first sulfate-free anionic surfactant has a concentration of from about 10 wt % to about 20 wt %.
Clause 22. A water-reduced shampoo composition of any one of clauses 16-21 wherein the first sulfate-free anionic surfactant has a concentration of from about 12 wt % to about 18 wt % relative to the total weight of the shampoo composition.
Clause 23. The water-reduced shampoo composition of any one of clauses 16-22 wherein the second sulfate-free anionic surfactant is selected from the group consisting of sodium cocoyl isethionate, sodium cocoyl methyl isethionate, sodium lauroyl isethionate, sodium lauroyl methyl isethionate, sodium lauroyl sarcosinate, sodium methyl oleoyl taurate.
Clause 24. The water-reduced shampoo composition of any one of clauses 16-23 wherein the second sulfate-free anionic surfactant is sodium lauroyl sarcosinate.
Clause 25. A water-reduced shampoo composition of any one of clauses 16-24 wherein the second sulfate-free anionic surfactant has a concentration of from about 5 wt % to about 8 wt % relative to the total weight of the shampoo composition.
Clause 26. A water-reduced shampoo composition of any one of clauses 16-25 wherein the first sulfate-free anionic surfactant to the second sulfate-free anionic surfactant are in a wt % ratio of from about 1.8:1 to about 4:1, preferably about 2:1 to about 3:1.
Clause 27. The water-reduced shampoo composition of any one of clauses 16-26 further comprising a third sulfate-free anionic surfactant, and wherein each of the first, second and third sulfate-free anionic surfactants are different components.
Clause 28. The water-reduced shampoo composition of clause 27 wherein the third sulfate-free anionic surfactant is sodium cocoyl methyl isethionate or sodium methyl oleoyl taurate, preferably wherein the third sulfate-free anionic surfactant is sodium cocoyl methyl isethionate.
Clause 29. A water-reduced shampoo composition of clause 27 or 28 wherein the third sulfate-free anionic surfactant has a concentration of from about 5 wt % to about 8 wt %, relative to the total weight of the shampoo composition.
Clause 30. A water-reduced shampoo composition of any one of clauses 27 to 29 wherein the first sulfate-free anionic surfactant to the third sulfate-free anionic surfactant are in a wt % ratio of from about 1.6:1 to about 2:1.
Clause 31. A water-reduced shampoo composition of any one of clauses 16 to 30 wherein the surfactant component is essentially or completely free of a sulfate anionic surfactant.
Clause 32. The water-reduced shampoo composition of any one of clauses 16 to 31, wherein the weight percentage ratio of anionic surfactants to non-ionic surfactant(s) is about 8:1 to about 12:1.
Clause 33. The water-reduced shampoo composition of any one of clauses 16 to 32, wherein the non-ionic surfactant comprises lauroyl/myristyl methyl glucamide and/or capryloyl/caproyl methyl glucamide.
Clause 34. The water-reduced shampoo composition of any one of clauses 16 to 33, wherein the non-ionic surfactant has a concentration of from about about 2 wt % to about 5 wt %, relative to the total weight of the shampoo composition.
Clause 35. The water-reduced shampoo composition of any one of clauses 16 to 34, wherein the composition is substantially free of amphoteric surfactants.
Clause 36. The water-reduced shampoo composition of any one of clauses 16 to 35 wherein the polyol is sorbitol.
Clause 37. The water-reduced shampoo composition of any one of clauses 16 to 35 wherein the polyol is a dihydric polyol selected from the group consisting of 1,2,3-butanetriol, ethylene glycol, diethylene glycol, 1,2 propylene diol, 1,3 propylene diol and any combination thereof; and preferably the dihydric polyol is selected from the group consisting of 1,2-propanediol, 1,3 propanediol and any combination thereof.
Clause 38. The water-reduced shampoo composition of any one of clauses 16 to 37 comprising the liquid polyol at a weight percentage concentration of from about 45 wt % to about 65 wt %, preferably from about 50 wt % to about 60 wt %.
Clause 39. The water-reduced shampoo composition of any one of clauses 16 to 38 wherein the third sulfate-free anionic surfactant is a taurate surfactant.

### Method for Use

Embodiments of the shampoo composition according to the invention may be applied to scalp hair through a method involving application of an aliquot of the concentrated shampoo composition directly to the hair or pre-diluting an aliquot of the concentrated composition with water followed by application to the hair. When an aliquot of the shampoo composition is applied directly, the aliquot may be distributed at least in part throughout the hair by massaging with fingers or a brush followed by dilution with water and spreading throughout the hair by rubbing and/or massaging with fingers. Working the portion of diluted composition through the hair for a period of at least 20 seconds establishes foaming action, distributes the shampoo, and delivers the cleaning components as needed. The shampoo may be rinsed from the hair by flooding the hair with flowing warm water while rubbing the hair. The rinsed hair may be towel dried, followed by drying with a warm hair dryer while combing.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified.

It has been surprisingly discovered that the shampoos compositions of the present disclosure, and in particular shampoo compositions containing a combination of at least two non-sulfate based anionic and non-ionic surfactants, a polyol (preferably glycerol and/or propanediol) and a certain/small amount of water does not only allow to formulate compact and significantly water reduced shampoos enabling an application amount reduction of ¾ to ⅔ vs. a conventional water-based shampoo, but without the typical compacted shampoo disadvantages related to rinseability speed, and product performance, it does also beat conventional, sulfate-surfactant based shampoo especially with regards to rinseability speed.

Half head tests done by stylists show these hair conditioning performance advantages of the water reduced shampoo according to the present invention in comparison to a conventional reference/market shampoo with high water level and even sulfate based surfactants. Half-head comparisons enable the hairstylist to evaluate the effects of hair products in comparison with a defined standard. This involves a sample of the test product being applied to the head of one model, and then being directly compared to a comparison sample or the untreated hair according to various technical hairstyling criteria. This test is termed a half-head comparison because the test and reference samples are applied to one half of the head respectively, thus enabling a direct comparison under absolutely identical test conditions (identical hair structure, degree of damage, hair colour etc.). The performance of the water reduced shampoo according to the present invention (examples 1, 2 and 3) was compared with the performance of a conventional, sulfate-surfactant based reference shampoo. The application amount weight ratio of example 1-3 to the reference shampoo was 0.25, the absolute application amount was adapted to the hair length of the models ranging from 1-2 gram of example 1 and 4-8 gram of the reference shampoo respectively.

The following hair care criteria were judged by experienced stylists: rinseability, measured as time needed for complete the shampoo wash off, wet combability, dry combability, dry feel and anti-frizz effect.

### Rinseability Measurement Protocol:

When carrying out the half-head comparison, make sure that the left and right sides are treated separately, always taking the center parting into consideration.

Ensure water force is set not too strong and water flow should be directed towards the side being rinsed.

Add a small amount of water to emulsify the product mass.

Position the shower head with flowing water at the center face line.

Use side of right hand rinse off the product on face line and the top (crown) area to the sideburn.

### Rinse the Middle and Tips of Hair

Important: If working on long hair, pile one side high at a time, so that each side can be rinsed.

Remove Clip from Section of Hair to be Rinsed.

After rinsing, lightly press the hair dry with a towel, taking the center parting into consideration.

Rinsing time measured is determined by the time required to rinse off the product completely on each hair section. The time required is measured with a stop watch and gives the evaluation of a longer or shorter rinsing time.

The rinsing time was determined among 4 test persons, examples 1, 2 and 3 were tested vs. the numbers indicated for how many test persons each criterion was judged how:

| | | Required |
|---|---|---|
| | Required | rinsing |
| | rinsing | time / s |
| | time / s | Examples |
| Test Leg | Reference | 1-3 |
| 2 g of example 1 vs. 8 g of reference SH | 70 | 45 |
| 2 g of example 1 vs. 6 g of reference SH | 70 | 50 |
| 1 g of example 2 vs. 4 g of reference SH | 54 | 32 |
| 2 g of example 3 vs. 8 g of reference SH | 70 | 40 |
| Average rinsing time ± standard deviation: | 66.0 ± 8.0 | 41.8 ± 7.7 |

| statistical significance (t-test, if >/=95%: | 99.5% |
|---|---|
| significant difference) of rinsing time | |
| difference between reference and examples | |
| 1-3: | |

### Reference Shampoo Composition:

82.4% water, 7.9% sodium lauryl ethersulfate, 5.7% sodium lauryl sulfate, 2.1% Cocamidopropyl Betaine, 0.5% perfume, 0.5% citric acid, 0.45% sodium benzoate, 0.2% salicylic acid, 0.15% EDTA, 0.1% methylparaben

The results indicated clearly that the water reduced compact shampoo according to the present invention leads to a statistically significant 37% easier/faster rinsing in comparison to a conventional, no water reduced shampoo.

Additionally, half head performance tests of the water reduced compact shampoo according to the present invention (example 1) versus the conventional, no water reduced reference shampoo were performed. The following hair care criteria were judged by experienced stylists: wet combability (easier/better or worse combability of wet hair), dry combability (easier/better or worse combability of the washed hair after drying), dry hair feel (softer/better or worse/rougher hair feel) and anti-frizz effect (if better/less frizzy hair or worse/more frizz). The respective half head test were carried out with 2 test persons, the numbers indicated for how many test persons each criterion was judged how:

| | Example 1 | Example 1 | Example 1 |
|---|---|---|---|
| | better than | equal than | worse than |
| | reference | reference | reference |
| Criterion | shampoo | shampoo | shampoo |
| Wet Combability | 2 | — | — |
| Dry Combability | 1 | — | — |
| Dry Hair Feel | 2 | — | — |
| Less Frizz | 1 | — | — |

The results indicated clearly that the water reduced compact shampoo according to the present invention leads even to better hair conditioning performance in comparison to a conventional, no water reduced shampoo.

Net—the shampoo according to the present invention has not only an unexpected and much higher rinsing effectiveness than average water-based based shampoos, it does also deliver a better conditioning performance.

### Example 1

| Raw Material | Amount | |
|---|---|---|
| Glycerin | 59 | g |
| Sodium Cocoyl Isethionate | 17.4 | g |
| Capryloyl/Caproyl Methyl Glucamide (and) | 3.0 | g |
| Lauroyl/Myristoyl Methyl Glucamide | | |
| Sodium Methyl Oleoyl Taurate | 2.6 | g |
| Sodium Lauroyl Sarcosinate | 0.95 | g |
| Citric Acid | 0.4 | g |
| Perfume | 0.8 | g |
| Water | 15.85 | g |

### Example 2

| Raw Material | Amount | |
|---|---|---|
| Glycerin | 57.2 | g |
| Sodium Cocoyl Isethionate | 17.4 | g |
| Capryloyl/Caproyl Methyl Glucamide (and) | 3.0 | g |
| Lauroyl/Myristoyl Methyl Glucamide | | |
| Sodium Methyl Oleoyl Taurate | 2.6 | g |
| Sodium Lauroyl Sarcosinate | 0.95 | g |
| Linoleamidopropyl PG-Dimonium Chloride Phosphate | 1.8 | g |
| Citric Acid | 0.4 | g |
| Perfume | 0.8 | g |
| Water | 15.85 | g |

### Example 3

| Raw Material | Amount | |
|---|---|---|
| Glycerin | 58.3 | g |
| Sodium Cocoyl Isethionate | 17.4 | g |
| Capryloyl/Caproyl Methyl Glucamide (and) | 3.0 | g |
| Lauroyl/Myristoyl Methyl Glucamide | | |
| Sodium Methyl Oleoyl Taurate | 2.6 | g |
| Sodium Lauroyl Sarcosinate | 0.95 | g |
| Polyquaternium-6 | 0.9 | g |
| Citric Acid | 0.4 | g |
| Perfume | 0.8 | g |
| Water | 15.85 | g |

### Example 4

| Raw Material | Amount | |
|---|---|---|
| Glycerin | 57 | g |
| Sodium Cocoyl Isethionate | 17.4 | g |
| Sodium Lauroyl Sarcosinate | 5.7 | g |
| Lauroyl/Myristoyl Methyl Glucamide | 2.1 | g |
| Citric Acid | 0.4 | g |
| Perfume | 0.8 | g |
| Water | 16.6 | g |

### Example 5

| Raw Material | Amount | |
|---|---|---|
| Glycerin | 59 | g |
| Sodium Cocoyl Methyl Isethionate | 18.5 | g |
| Capryloyl/Caproyl Methyl Glucamide (and) | 3.0 | g |
| Lauroyl/Myristoyl Methyl Glucamide | | |
| Sodium Methyl Oleoyl Taurate | 2.6 | g |
| Sodium Lauroyl Sarcosinate | 0.95 | g |
| Citric Acid | 0.4 | g |
| Perfume | 0.6 | g |
| Water | 14.95 | g |

### Example 6

| Raw Material | Amount | |
|---|---|---|
| Glycerin | 57 | g |
| Sodium Cocoyl Isethionate | 17.4 | g |
| Sodium Lauroyl Sarcosinate | 5.7 | g |
| Lauroyl/Myristoyl Methyl Glucamide | 2.1 | g |
| Hydroxypropyl Guar Hydroxypropyltrimonium Chloride | 0.6 | g |
| Citric Acid | 0.4 | g |
| Perfume | 0.8 | g |
| Water | 16.2 | g |

### Example 7

| Raw Material | Amount | |
|---|---|---|
| Glycerin | 59 | g |
| Sodium Cocoyl Methyl Isethionate | 18.5 | g |
| Capryloyl/Caproyl Methyl Glucamide (and) | 3.0 | g |
| Lauroyl/Myristoyl Methyl Glucamide | | |
| Sodium Methyl Oleoyl Taurate | 2.6 | g |
| Sodium Lauroyl Sarcosinate | 0.95 | g |
| Citric Acid | 0.4 | g |
| Perfume | 0.6 | g |
| Acid Violet 43 | 0.15 | g |
| Water | 14.95 | g |

### Example 8

| Raw Material | Amount | |
|---|---|---|
| Glycerin | 57 | g |
| Sodium Cocoyl Isethionate | 17.4 | g |
| Sodium Lauroyl Sarcosinate | 5.7 | g |
| Lauroyl/Myristoyl Methyl Glucamide | 2.1 | g |
| Pirocton Olamine | 1.0 | g |
| Hydroxypropyl Guar Hydroxypropyltrimonium Chloride | 0.6 | g |
| Citric Acid | 0.4 | g |
| Perfume | 0.8 | g |
| Water | 16.2 | g |

### Example 9

| Raw Material | Amount | |
|---|---|---|
| Glycerin | 47 | g |
| Sodium Laureth Sulfate | 31 | g |
| Polyquaternium-10 | 0.6 | g |
| Perfume | 0.4 | g |
| Water | 21 | g |

### Example 10

| Raw Material | Amount | |
|---|---|---|
| Glycerin | 54 | g |
| Sodium Laureth Sulfate | 31 | g |
| Hydroxypropyl Guar Hydroxypropyltrimonium Chloride | 0.6 | g |
| Perfume | 0.4 | g |
| Water | 14 | g |

### Example 11

| Raw Material | Amount | |
|---|---|---|
| Glycerin | 54 | g |
| Sodium Cocoyl Isethionate | 12.5 | g |
| Capryloyl/Caproyl Methyl Glucamide (and) | 3.0 | g |
| Lauroyl/Myristoyl Methyl Glucamide | | |
| Sodium Cocoyl Methyl Isethionate | 7.5 | g |
| Sodium Lauroyl Sarcosinate | 7.0 | g |
| Citric Acid | 2.2 | g |
| Perfume | 0.8 | g |
| Water | 13 | g |

### Miscellaneous Statements

The invention has been described broadly and generically herein. Each of the narrower species and subgeneric groupings falling within the generic disclosure also form part of the invention. This includes the generic description of the invention with a proviso or negative limitation removing any patient matter from the genus, regardless of whether or not the excised material is specifically recited herein. The inventions, examples, results and statement of embodiments described, stated and claimed herein may have attributes and embodiments include, but not limited to, those set forth or described or referenced in this application.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed and as provided by the statements of embodiments. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims and the statements of embodiments.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, text book or other referenced material or document.

The written description of this patent application includes all claims, examples and statements of embodiments. All claims and statements of embodiments including all original claims are hereby incorporated by reference in their entirety into the written description portion of the specification and the right is reserved to physically incorporated into the written description or any other portion of the application any and all such claims and statements of embodiments. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims and the statements of embodiments. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims and the statements of embodiments.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation.

The methods and processes illustratively described herein suitably may be practiced in differing orders of steps, and that they are not necessarily restricted to the orders of steps indicated herein or in the claims.

Unless defined otherwise, all technical and scientific terms used herein have the same meaning as commonly understood by a person of ordinary skill in the art.

1. A water-reduced shampoo composition comprising:
i) a liquid polyol at a weight percentage concentration of from about 40 wt % to about 80 wt %, preferably from about 45 wt % to about 80 wt %;
wherein the polyol comprises at least one of a polyhydric, trihydric or dihydric alcohol, or any combination thereof; the polyhydric alcohol being a polyhydroxyl C4-C8 alkane; the dihydric alcohol being a dihydroxyl C2-C8 alkane; and the trihydric alcohol being a trihydroxyl C3-C8 alkane;
ii) a surfactant component at a weight percentage concentration of from about 10 wt % to about 40 wt %, preferably from about 15 wt % to about 35 wt %, more preferably from about 20 wt % to about 30 wt %;
wherein the surfactant component comprises at least one or more non-ionic surfactants, at least a first sulfate-free anionic surfactant, and a second sulfate-free anionic surfactant; and
wherein the first sulfate-free anionic surfactant and the second sulfate-free anionic surfactant are different components;
iii) water at a weight percentage concentration of from about 5 wt % to about 25 wt %;
wherein all weight percentages are relative to the total weight of the composition.

2. The water-reduced shampoo composition of claim 1 wherein the polyol comprises a trihydric alcohol.

3. The water-reduced shampoo composition of claim 2, wherein the trihydric alcohol is selected from the group consisting of glycerin (propyl triol), 1,2,3-butylene triol, 1,2,5-pentylene triol, 1,3,6-hexylene triol and any combination thereof: preferably the trihydric alcohol is glycerin.

4. The water-reduced shampoo composition of claim 1 wherein the first sulfate-free anionic surfactant is an isethionate compound, preferably an acyl isethionate or a acyl methyl isethionate, at a weight percentage range of from about 5 wt % to about 25 wt % relative to the total weight of the composition.

5. The water-reduced shampoo composition of claim 4 wherein the isethionate compound is selected from the group consisting of sodium cocoyl isethionate, sodium cocoyl methyl isethionate, sodium lauroyl isethionate and sodium lauroyl methyl isethionate: preferably the isethionate compound is sodium cocoyl isethionate.

6. The water-reduced shampoo composition of claim 1 wherein the first sulfate-free anionic surfactant has a concentration of from about 5 wt % to about 20 wt %, preferably about 10 wt % to about 20 wt %, and most preferably about 12 wt % to about 18 wt %, relative to the total weight of the shampoo composition.

7. The water-reduced shampoo composition of claim 1 wherein the second sulfate-free anionic surfactant is selected from the group of acyl isethionates/methyl isethionates, acyl glycinates, acyl taurates, acyl amino acids, acyl sarcosinates, sulfosuccinates and sulfonates, wherein the acyl groups comprise from 6 to 30 carbon atoms, preferably wherein the second sulfate-free anionic surfactant is selected from the group consisting of sodium cocoyl isethionate, sodium cocoyl methyl isethionate, sodium lauroyl isethionate, sodium lauroyl methyl isethionate, sodium lauroyl sarcosinate, sodium methyl oleoyl taurate; and more preferably wherein the second sulfate-free anionic surfactant is sodium lauroyl sarcosinate.

8. The water-reduced shampoo composition of claim 1 wherein the second sulfate-free anionic surfactant has a concentration of from about 2 wt % to about 10 wt %, preferably about 5 wt % to about 8 wt %, relative to the total weight of the shampoo composition.

9. The water-reduced shampoo composition of claim 1 wherein the first sulfate-free anionic surfactant to the second sulfate-free anionic surfactant are in a wt % ratio of from about 1.5:1 to about 7:1, preferably about 1.8:1 to about 4:1, more preferably about 2:1 to about 3:1.

10. The water-reduced shampoo composition of claim 1 further comprising a third sulfate-free anionic surfactant, and wherein each of the first, second and third sulfate-free anionic surfactants are different components, preferably wherein the third sulfate-free anionic surfactant is an acyl/methyl isethionate or an acyl taurate.

11. The water-reduced shampoo composition of claim 10 wherein the third sulfate-free anionic surfactant is selected from the group consisting of sodium lauroyl sarcosinate, sodium cocoyl methyl isethionate, sodium lauroyl isethionate, sodium lauroyl methyl isethionate, sodium methyl oleoyl taurate; and preferably wherein the third sulfate-free anionic surfactant is sodium cocoyl methyl isethionate or sodium methyl oleoyl taurate, more preferably wherein the third sulfate-free anionic surfactant is sodium cocoyl methyl isethionate.

12. The water-reduced shampoo composition of claim 10 wherein the third sulfate-free anionic surfactant has a concentration of from about 2 wt % to about 10 wt %, preferably from about 5 wt % to about 8 wt %, relative to the total weight of the shampoo composition.

13. The water-reduced shampoo composition of claim 10 wherein the first sulfate-free anionic surfactant to the third sulfate-free anionic surfactant are in a wt % ratio of from about 1.3:1 to about 7:1, preferably from about 1.5:1 to about 4:1, more preferably from about 1.6:1 to about 3:1.

14. The water-reduced shampoo composition according to claim 1 wherein the surfactant component is essentially or completely free of a sulfate anionic surfactant.

15. The water-reduced shampoo composition of claim 1, wherein the weight percentage ratio of anionic surfactants to non-ionic surfactant(s) is from about 5:1 to about 15:1, preferably about 8:1 to about 12:1.

16. The water-reduced shampoo composition of claim 1, wherein the non-ionic surfactant comprises one or more of lauroyl/myristyl methyl glucamide, capryloyl/caproyl methyl glucamide, capryloyl/caproyl glucamide, lauryl glucoside, decyl glucoside, coco glucoside, and any combination thereof, preferably wherein the non-ionic surfactant comprises lauroyl/myristyl methyl glucamide and/or capryloyl/caproyl methyl glucamide.

17. The water-reduced shampoo composition of claim 10, wherein the non-ionic surfactant comprises one or more of lauroyl/myristyl methyl glucamide, capryloyl/caproyl methyl glucamide, capryloyl/caproyl glucamide, lauryl glucoside, decyl glucoside, coco glucoside, and any combination thereof, preferably wherein the non-ionic surfactant comprises lauroyl/myristyl methyl glucamide and/or capryloyl/caproyl methyl glucamide.

18. The water-reduced shampoo composition of claim 1, wherein the non-ionic surfactant has a concentration of from about 1 wt % to about 10 wt %, preferably from about 2 wt % to about 5 wt %, relative to the total weight of the shampoo composition.

19. The water-reduced shampoo composition of claim 1, wherein the first sulfate-free anionic surfactant is an isethionate compound, preferably sodium cocoyl isethionate, the second sulfate-free anionic surfactant is sodium lauroyl sarcosinate, and the non-ionic surfactant comprises lauroyl/myristyl methyl glucamide and/or capryloyl/caproyl methyl, and optionally further comprising a third sulfate-free anionic surfactant, wherein the third sulfate-free anionic surfactant is sodium cocoyl methyl isethionate, and wherein each of the first, second and third sulfate-free anionic surfactants are different components.

20. The water-reduced shampoo composition of claim 19, wherein the first sulfate-free anionic surfactant is an isethionate compound at a weight percentage range of from about 10 wt % to about 20 wt %, the second sulfate-free anionic surfactant has a concentration of from about 5 wt % to about 8 wt %, and the non-ionic surfactant comprises lauroyl/myristyl methyl glucamide and/or capryloyl/caproyl methyl and has a concentration of from about about 2 wt % to about 5 wt % relative to the total weight of the shampoo composition, and optionally wherein the third sulfate-free anionic surfactant has a concentration of from about 5 wt % to about 8 wt %, relative to the total weight of the shampoo composition.

21. The water-reduced shampoo composition of claim 1, wherein the composition is substantially free of amphoteric surfactants.

22. The water-reduced shampoo composition of claim 1 wherein the polyol is selected from the group consisting of erythritol, xylitol, sorbitol, propylene triol, 1,2,3-butylene triol, 1,2,5-pentylene triol, 1,3,6-hexylene triol, ethylene glycol, 1,2-propylene diol, 1,3 propane diol, 1,2-butane diol, 1,3-butane diol, 1,2 pentane diol, diethylene glycol, dipropylene glycol and any combination thereof; preferably the polyol is selected from the group consisting of sorbitol, glycerin (propyl triol), 1,2,3-butanetriol, ethylene glycol, diethylene glycol, 1,2 propylene diol, 1,3 propylene diol and any combination thereof; and more preferably the polyol is selected from the group consisting of sorbitol, glycerin, 1,2-propanediol, 1,3 propanediol and any combination thereof.

23. The water-reduced shampoo composition of claim 1 comprising the liquid polyol at a weight percentage concentration of from about 45 wt % to about 65 wt %, preferably from about 50 wt % to about 60 wt %.

24. The water-reduced shampoo composition of claim 1, wherein the composition further comprises a conditioning agent; preferably the concentration of the conditioning agent is in a range of from about 0.05 wt % to about 10 wt %, more preferably from about 0.1 wt % to about 5 wt % relative to the total weight of the shampoo composition; and especially more preferably the conditioning agent comprises at least a quaternary ammonium organic compound.

25. Use of the water-reduced shampoo composition of claim 1 for reducing the use of rinse water.
